(19) United States
(12) Patent Application Publication
(10) Pub. No.: US 2008/0188761 A1
(43) Pub. Date: Aug. 7, 2008

(54) ECG-BASED IDENTIFICATION OF IMPAIRED IKR KINETICS

(76) Inventor: Jean-Philippe Couderc, Rochester, NY (US)

Correspondence Address: JAECKLE FLEISCHMANN & MUGEL, LLP, 190 Linden Oaks, ROCHESTER, NY, 14625-2812, US

(21) Appl. No.: 11/939,374

(22) Filed: Nov. 13, 2007

Related U.S. Application Data
(60) Provisional application No. 60/865,572, filed on Nov. 13, 2006.

(51) Int. Cl. A61B 5/0402

(52) U.S. Cl. 600/509

(57) ABSTRACT

A method of assaying impaired delayed rectifier potassium current function is disclosed. One or more electrocardiogram (ECG) signals are obtained. One or more heart rates corresponding to a plurality of beats of the one or more ECG signals are obtained. One or more T-wave amplitudes are determined corresponding to one or more beats of the one or more ECG signals. A determination is made whether the one or more T-wave amplitudes as a function of the one or more heart rates are relatively independent of the corresponding one or more heart rates, thereby indicating impaired delayed rectifier potassium current function. An associated biomarker, system, and method for determining the ability of a pharmacological agent to impair delayed rectifier potassium current function are also disclosed.

### RELATED APPLICATIONS

This application claims priority to U.S. provisional patent application 60/865,572, entitled “Loss of Heart Rate Dependency of T-Wave Amplitude as a Function of Impaired IKr Kinetics, and Methods and Uses Thereof”, and filed Nov. 13, 2006. The entire 60/865,572 patent application is hereby officially incorporated by reference.

### FIELD

The claimed invention relates to electrocardiogram-based analysis systems and methods. More particularly, the claimed invention relates to ECG-based identification of impaired IKr kinetics.

### BACKGROUND

The electrocardiogram (ECG) is based on the electrical activity of the heart muscle cells. In the resting stage, the inside of the cardiac cells has a negative charge compared to the outside of the cells. The resulting voltage difference between the internal and the external spaces of the cell membrane is called transmembrane potential. The discharging of this voltage is known as depolarization and is associated with the start of the contraction of the heart muscle cell fibers. After contraction of the ventricles, the heart muscle cells redevelop substantially the same voltage over the cell membrane. This recovery phase is called the repolarization process of the heart ventricles. An ECG measured from the skin surface measures a total electrical component created by the depolarization and repolarization of the heart's muscular cells.

The repolarization of the heart is made possible in part by ion channels within the myocardial cells of the heart which allow an ion current to redistribute charge. It is highly important that the regulation of the ion currents during the ventricular repolarization process occurs without interference, since a delay in this process or any other abnormalities can lead to a substantially increased risk for sudden cardiac death.

Medical professionals have used electrocardiograms (ECG's) to examine the ventricular repolarization period, also known as the QT interval, to check for elongation of the QT interval. In general, an elongated QT interval may be considered to be indicative of a delay in the ventricular repolarization process. While medications in some cases may be the cause of an elongated QT interval, for many people are predisposed to have an elongated QT interval due to one or more congenital mutations. Those patients having a congenital predisposition for an elongated QT interval are referred-to as having Long QT Syndrome, or LQTS. The clinical course and the precipitating risk factors in the congenital Long QT Syndrome (LQTS) are genotype specific. Among LQTS mutations, KvLQT1 (LQT1) and HERG (LQT2) mutations have the higher likelihood of recurrent cardiac events and their diagnosis is crucial to reduce lethal outcome.

The Long QT Syndrome (LQTS) is an inherited disease caused by genetically determined defects in trans-membrane ion channel subunit. LQTS patients are at high risk of sudden cardiac death due to the development of ventricular tachycardia degenerating in ventricular fibrillation and cardiac arrest. The prevalence of the syndrome may be expected to occur in 1 in 3000-5000 individuals per year in the United States. The number of cases of sudden cardiac death associated with the LQTS is unknown but among the 300,000 sudden cardiac deaths documented each year, one may expect around 2-5% having LQTS-related arrhythmic death. In the US, the syndrome remains an under diagnosed disorder because an estimated 10 to 15% of the LQTS gene carrier patients have a QT interval duration near normal values.

Seven mutant genes have been associated with LQTS: KvLQT1/minK, HERG, SCN5A, Ankyrin B, KCNE1, KCNE2 and KCNJ2. Most of these genes encode cardiac ion channels and their mutation leads to dysfunction of the ion current kinetics. Among the current 150 mutations identified in the seven LQTS genes, LQT1 and LQT2 represent the majority of cases (88%). The mutation of the HERG (LQT2) gene decreases the rapidly activating delayed rectifier potassium (K+) current (IKr). The inhibition of IKr ion current is associated with a prolongation of the action potentials within the heart leading in general to a prolonged QT interval on the surface ECGs. In addition to the measurement of a QT interval or QT interval prolongation, the related prolongation of QT interval as corrected for variation as a function of heart rate, i.e., QTc interval prolongation (collectively “QT/QTc interval prolongation”) may also be determined.

While the use of QT/QTc interval prolongation is commonly accepted as being useful for assaying an increased risk for arrhythmic events (see, e.g., the E14 protocol “Clinical Evaluation of QT/QTc Interval Prolongation and Proarrythmic Potential for Non-Antiarrhythmic Drugs, U.S. Department of Health and Human Services, Food and Drug Administration, October, 2005), this metric is not always predictive of such events. For example, prolongation of the QT interval is not found in 10 to 15% of patients with the congenital form of the long QT syndrome (“LQTS”) carrying a HERG. Similarly, in the acquired form of the LQTS, polymorphic ventricular tachycardia—but no strong QT prolongation—are documented in individuals who have received drugs that block the delayed rectifier potassium current (IKr). Thus there is a need for other metrics for identifying risk of arrhythmic events which result from the impairment of IKr kinetics.

### SUMMARY

A method of assaying impaired delayed rectifier potassium current function is disclosed. One or more electrocardiogram (ECG) signals are obtained. One or more heart rates corresponding to a plurality of beats of the one or more ECG signals are obtained. One or more T-wave amplitudes are determined corresponding to one or more beats of the one or more ECG signals. A determination is made whether the one or more T-wave amplitudes as a function of the one or more heart rates are relatively independent of the corresponding one or more heart rates, thereby indicating impaired delayed rectifier potassium current function.

An associated biomarker is also disclosed.

A system for assaying impaired delayed rectifier potassium current function is also disclosed. The system has a processor configured to determine an indication of impaired delayed rectifier potassium current function based on a comparison of T-wave amplitudes from ECG data with corresponding heart rates for the T-wave amplitudes. The system also has a data input coupled to the processor and configured to provide the processor with the ECG data. The system further has a user interface coupled to either the processor or the data input.

A method for determining the ability of a pharmacological agent to impair delayed rectifier potassium current function is further disclosed. A baseline electrocardiogram (ECG) is obtained for a mammal at different heart rates. A baseline dependency of a plurality of T-wave amplitudes on corresponding heart rates for the baseline ECG is determined. The pharmacological agent is administered to the mammal. A follow-on ECG is obtained for the mammal at different heart rates. A follow-on dependency of a plurality of T-wave amplitudes on corresponding heart rates for the follow-on ECG is determined. The follow-on dependency is compared with the baseline dependency.

### BRIEF DESCRIPTION OF THE DRAWINGS

FIG. 1A schematically illustrates an embodiment of an ECG showing one heart beat and some of the labels which are commonly assigned to various portions of the ECG signal.

FIG. 1B schematically illustrates another embodiment of an ECG showing one heart beat and some scalar metrics which may be determined for various portions of the ECG signal.

FIG. 2A illustrates experimental data showing QT interval distribution across heart rate in healthy individuals on and off sotalol.

FIG. 2B illustrates experimental data showing QT interval distribution across heart rate in patients with congenital long QT syndrome.

FIG. 3A illustrates experimental data showing T-amplitude distribution across heart rate in healthy individuals on and off sotalol.

FIG. 3B illustrates experimental data showing T-amplitude distribution across heart rate in patients with congenital long QT syndrome.

FIGS. 4A-4C illustrate experimental data showing ECG tracings (lead II) for the same individual at similar heart rate during baseline and on single and double dose of sotalol.

FIG. 5A illustrates experimental data showing QT/RR relationship in healthy individuals, and in individuals with the acquired and congenital forms of the long QT syndrome.

FIG. 5B illustrates experimental data showing T-amplitude/RR relationship in healthy individuals, and in individuals with the acquired and congenital forms of the long QT syndrome.

FIG. 6 illustrates one embodiment of a method of assaying impaired delayed rectifier potassium current function.

FIG. 7 schematically illustrates one embodiment of a system for assaying impaired delayed rectifier potassium current function.

FIG. 8 schematically illustrates another embodiment of a system for assaying impaired delayed rectifier potassium current function.

FIG. 9 schematically illustrates a further embodiment of a system for assaying impaired delayed rectifier potassium current function.

FIG. 10 schematically illustrates another embodiment of a system for assaying impaired delayed rectifier potassium current function.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

A surface electrocardiogram (ECG) may be measured by an ECG capture device which can have one or more leads which are coupled to a person's body in various locations. The electrical activity occurring within individual cells throughout the heart produces a cardiac electrical vector which can be measured at the skin's surface by the ECG capture device leads. The signal registered at the skin's surface originates from many simultaneously propagating activation fronts at different locations, each of which affects the size of the total component. One type of ECG capture device is a twelve-lead signal device, although ECG capture devices of any number of leads may be used to gather a set of ECG signals for use in assessing repolarization abnormality.

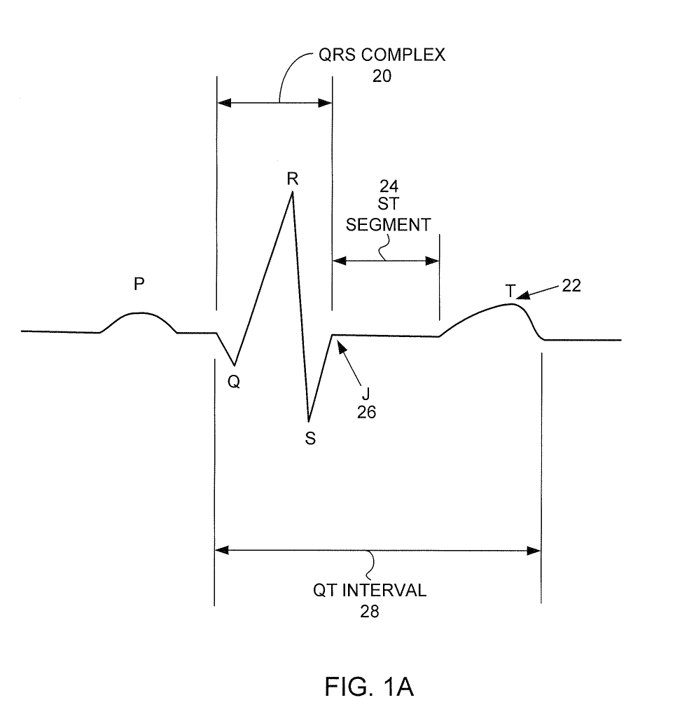

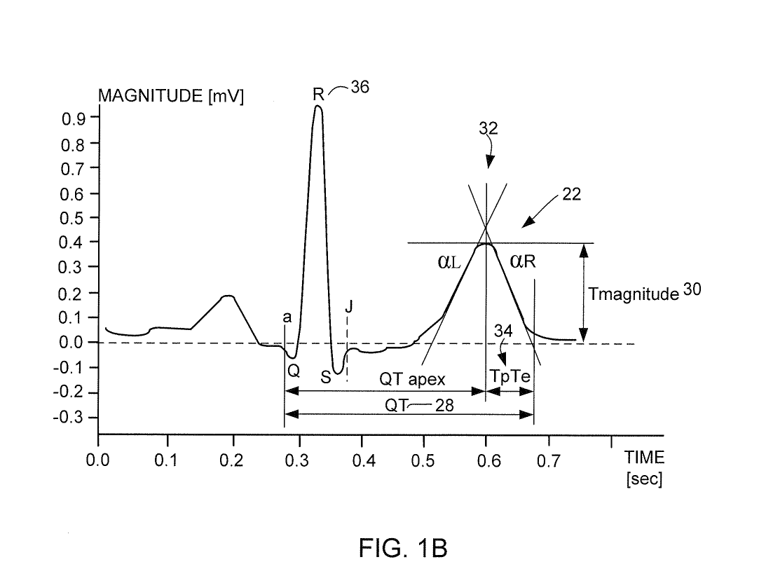

FIG. 1A schematically illustrates an embodiment of an ECG showing one heart beat and some of the labels which are commonly assigned to various portions of the ECG signal. The QRS complex 20 is associated with the depolarization of the heart ventricles. The QT interval 28 and the T-wave 22 are associated with repolarization of the heart ventricles. The ST segment 24 falls between the QRS complex 20 and the T-wave 22. The J point 26 is located where the QRS complex 20 joins the ST segment 24. For reference, the QT interval 28 discussed above, and which may be an indicator of Long QT Syndrome, is illustrated.

FIG. 1B schematically illustrates another embodiment of an ECG showing one heart beat and some scalar metrics which may be determined for various portions of the ECG signal. For reference, the QT interval 28 is illustrated again, as occurring from the start of the QRS complex and going until the end of the T wave 22. There are various ways of determining the end of the T-wave 22. Here, a right slope αR of the T-wave 22 is projected down to the baseline voltage, and where the right slope αR of the T-wave intersects the baseline voltage (in this case where the right slope αR of the T-wave intersects zero volts) determines the end of the T-wave 22. The T-wave 22 has a Tmagnitude 30 corresponding to the peak of the T-wave 22. The peak of the T-wave 22 occurs at a T-wave peak time 32. The time from the T-wave peak time 32 until the end of the T-wave is defined as the TpTe interval 34. The heartbeat duration RR is measured from one R peak 36 to a next R peak. A peak voltage during the QT interval (QTpeak) may be determined.

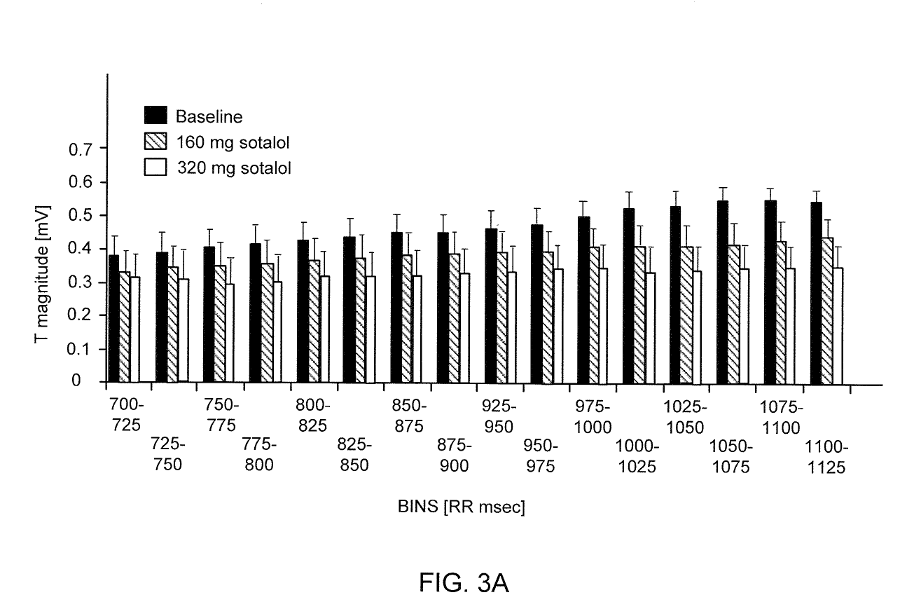

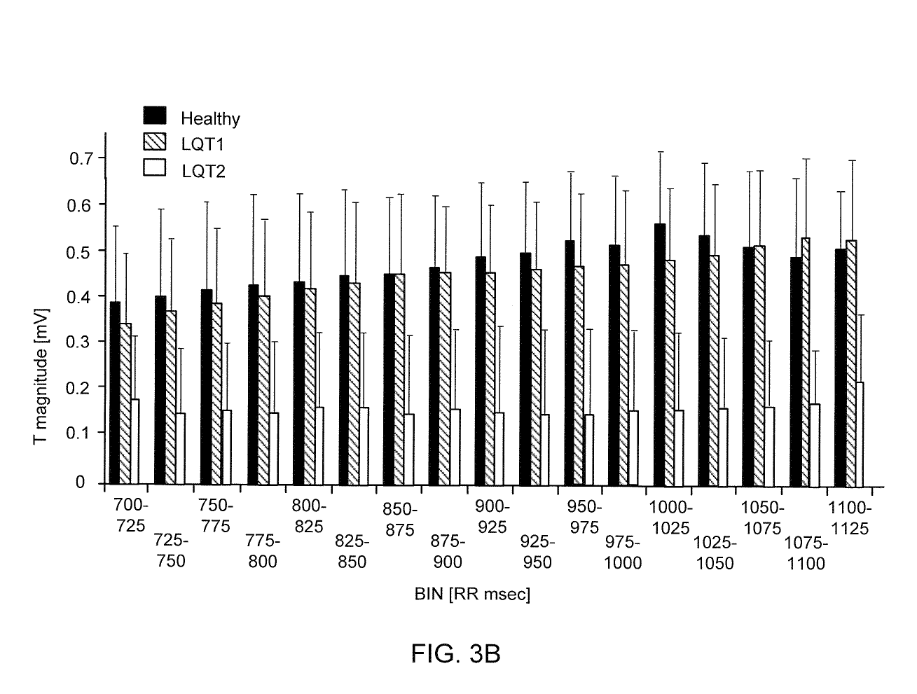

The claimed invention provides a new metric for arrhythmic event risk identification based on the loss of the dependency on heart rate of the T-wave amplitude under conditions of impaired IKr kinetics, as is demonstrated particularly in FIGS. 3A and 3B.

Specifically, the present invention is based on the novel determination that, in patients with impaired IKr kinetics resulting either from congenital defect (i.e., LQT2 carriers) or via drug administration (e.g., sotalol), the amplitude of the T-wave portion of the electrocardiogram (“ECG”) is relatively independent of heart rate. This situation is markedly different than that found for either healthy individuals or carriers of the LQT1 congenital defect, where in both cases T-wave amplitude increases as heart rate decreases. This situation is also different than is observed when change in QT interval is measured against heart rate (QT/RR; see particularly FIG. 1), where such change as a function of heart rate is much less pronounced than in T-wave amplitude vs. heart rate measurement.

These observations are expected to have utility in a variety of situations, including particularly a better ability to correlate surface electrical changes (i.e., ECG measurements) with changes that are occurring at the molecular level of heart function. Thus for example, the observations detailed herein may be particularly useful for assaying changes in IKr function (e.g., as result from mutations to ion channel function or modifications to such channel function resulting from drug binding, etc.) using T-wave vs. heart rate or other impairments of adaptation of T-wave amplitude to changing heart rate.

Given the importance of IKr function in the unexpected cardiotoxic effects of administered drugs, one application of such correlations is in predicting the cardiotoxic effects of drugs, including specifically a better understanding of drug effects as they specifically impact IKr kinetics, i.e., for separating drug effects on different molecular components of the heart. This predictive ability is expected to be particularly useful for drug development, where it may be particularly desirable to ensure no negative effect on IKr kinetics while less important to avoid effects on other components of the heart's electrophysiology.

Another example of the use of such correlative understanding between. genotype and phenotype is a more complete understanding of the degree to which genotypic changes in channels involved in IKr kinetics produce actual alterations in such kinetics, as assayed by surface electrical (i.e., phenotypic) changes. Thus, for example, the observations of the present invention may be used to probe the penetrance of, e.g., LQT2 mutations.

Other additional uses of the observations of the present invention include the extension of the metrics described herein to changes in other ion channel or physiological function, i.e., to the development of the non-QT-elongation metrics of the present invention for other establishment of other correlations between ECG and underlying genotypic/molecular changes and alterations in the electrophysiology of the heart and associated systems.

The QTc interval prolongation is not a perfect surrogate marker of the presence of an increased risk for arrhythmic events. In the search for alternative markers, we investigated the role of T-wave adaptation to heart rate changes for identifying abnormal repolarization in the congenital and drug-induced forms of the long QT syndrome (LQTS).

Our investigation is based on the analysis of 12-lead digital Holter recordings in: 49 LQT1 carriers, 25 LQT2 carriers, 37 healthy individuals off drugs and on 160 mg of sotalol, and 21 of them also on 320 mg of sotalol. The Holter recordings were used to investigate repolarization parameters and their heart rate dependency.

A loss of heart rate dependency of the T-wave amplitude was found as a common feature in individuals with impaired IKr kinetics: LQT2 carriers and subjects on sotalol. The Tamplitude/RR slope was significantly (p<0.05) flatter in LQT2 (0.31±0.27 pV/ms) than in both LQT1 (0.62±0.40 pV/ms) and healthy individuals (0.55±0.29 pV/ms). A reduction of the slope and dose-dependent effect of sotalol on the slope was observed (160 mg dose: 0.26±0.19 pV/ms; 320 mg dose: 0.21±0.14 pV/ms). The QT/RR slope was less effective than T amplitude/RR slope in differentiating studied groups.

Impaired adaptation of T-wave amplitude to changing heart rate is a common electrocardiographic feature associated with HERG mutation and IKr blocking by sotalol. This ECG marker may play an important role in the future of assessment of both the penetrance of HERG-mutation and the IKr-related cardiotoxicity of drugs.

In the acquired long QT syndrome (LQTS), an inhibition of the rapidly activating delayed rectifier potassium current (IKr) is the main mechanism associated with drug-induced repolarization delay (1). The molecules of these QT-prolonging drug bind to various sites of the KCNH2/HERG channels of the myocardial cells and disturb their functional properties. The underlying torsadogenic mechanism observed in drug-induced LQTS is similar to the one reported in patients with the LQT2 syndrome. In this congenital form of the syndrome, the genetic (KCNH2/HERG) mutation leads to structural ion defects or/and intra cellular “trafficking” abnormalities causing a reduction in the number of operational IKr specific ion channels and thus delaying the entire ventricular repolarization process.

It is well-accepted that a QTc interval beyond 500 msec is a clear predisposing factor for the occurrence of torsades de pointes (TdPs) in both congenital and acquired form of LQTS, but the presence of a QT-interval duration below 500 msec is associated with an uncertain outcome. The majority of patients with acquired or congenital LQTS show QTc<500 ms the value with limited diagnostic and prognostics significance. The development of new biomarkers complementing the QT interval measurements for the identification of a predisposition to ventricular arrhythmias in congenital LQTS and for the assessment of the IKr inhibitory effect of drugs is of major interest.

Repolarization is a dynamic phenomenon largely depending on heart rate. The QT/RR relationship and QT hysteresis phenomenon have been investigated over the past few years indicating that QT/RR slope could be considered as a marker of sudden cardiac death after myocardial infarction, and demonstrating that QT hysteresis is strongly modified by IKr blocking compounds. In this study, we aimed to investigate the dynamic aspects of the repolarization morphology, namely T wave amplitude adaptation to changing heart rate, during various conditions attributed to abnormal IKr kinetics due to congenital or acquired forms of LQTS.

### Experimental Data

The study populations consisted of: 49 LQT1 carriers, 25 LQT2 carriers, and 37 healthy individuals who participated in a study focused on effect of sotalol on ECG parameters of repolarization. The group of LQTS patients included individuals with genetically identified KCNH2 (HERG) and KCNQ1 mutations, we investigated these two types of mutations since they are associated with two different ion-kinetics dysfunctions of potassium currents: the KCNH2 have been linked to a reduction of the rapidly activating repolarizing potassium current (IKr) and the KCNQ1 to the slowly activating ion current IKs. We analyzed the patients in whom digital 24-hour Holter recordings were available in order to be able to investigate heart rate dependency of their repolarization morphology. The protocol of this study included 3 days of Holter recordings: at baseline, on single 160 mg dose of sotalol and a 320 mg dose of sotalol.

### ECG Recordings and Processings

All the Holter ECGs were recorded using the same type of equipment: 24-hour Holter recorder H12 (H-12 recorder, Mortara Instrument, Milwaukee, Wis.) providing digital ECG signal at a sampling frequency of 180 Hz and with 12 bit amplitude resolution (6.25 pv). In this recording, 8 original leads are recorded and the remaining 4 leads (augmented limb leads aVR, aVL, aVF and lead III) are computed. Beat classification was performed using the HScribe scanning software (HScribe, Mortara Instrument, Milwaukee, Wi.) and manually reviewed and adjusted when necessary. The information about cardiac beat annotation was exported in XML format and integrated into the COMPAS software (COMPhrensive Analysis of the repolarization Segment software, University of Rochester Medical Center Rochester, N.Y.) for the analysis of repolarization morphology.

### Ensuring Repolarization Stability: Steady State

To ensure stability of repolarization and increase signal-to-noise ratio, we adopted the following strategy: the Holter recordings were divided in sections of 10 continuous sinus beats. Only the sections preceded by stable heart rate within the previous 5 minutes were selected for the analysis. Heart rate stability required having less than 300 ms changes between continuous RR values within the 5 minutes. Then, the remaining 10-beat sections were analyzed if their variation of RR intervals remained inferior to ±10% of their average RR intervals. The QT interval and T-amplitude measurements were based on median beats computed from each so-called “stable representative beats”.

### Measuring QT Interval and T-Wave Amplitude

Based on a technique ensuring an appropriate stability of the modeling of the relationship between repolarization measurements and the RR intervals disclosed in a publication by Couderc J P, Xiaojuan X, Zerba W, Moss A J, entitled, “Assessment of the Stability of the Individual-Based Correction of QT Interval for Heart Rate”, published in the Ann. Noninvasive. Electrocardiol. 2005 January; 10(1):25-34, and herein officially incorporated by reference, we investigated two electrocardiographic measurements: the QT interval and the amplitude of the T-wave and their relationship with RR intervals.

The QT interval and T amplitude were measured on the stable representative beats from lead II. The end of T-wave was identified based on the tangent method, the crossing point between the tangent and the isoelectric line identifying the end of the T-wave, although other methods of measuring the QT interval could be used in other embodiments. The amplitude of the T-wave was measured at the maximum value of the T-wave signal in absolute value. For negative T-waves, the sign of the amplitude was conserved. For biphasic T-waves, we measured the highest portion of the signal in absolute value. No manual adjustments of measurements were performed. Since sotalol protocol provided ECG data on drug only during day hours, only diurnal periods from 9:00 AM till 7:00 PM were included.

### RR Bin Analysis of the T-Wave Amplitude and QT Interval

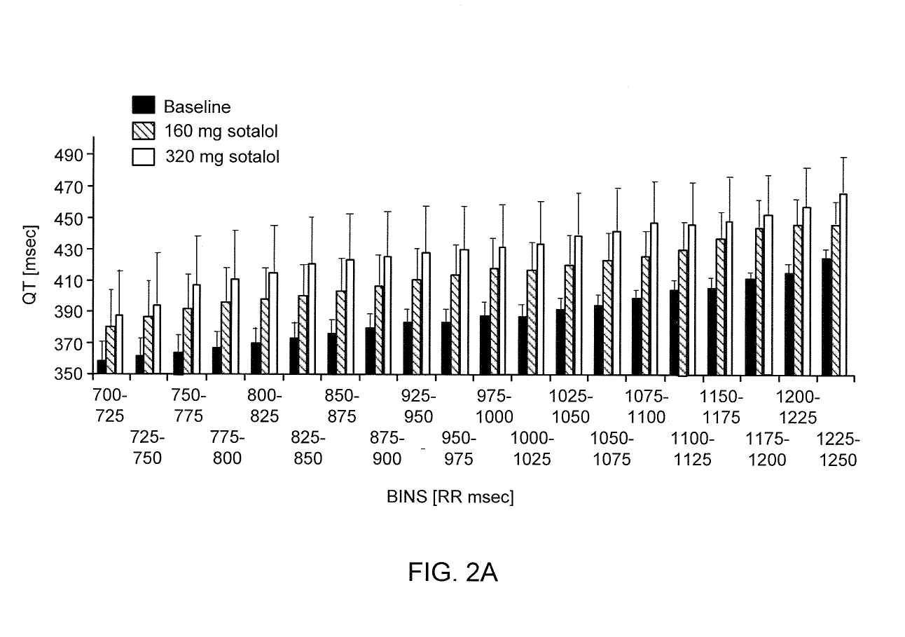

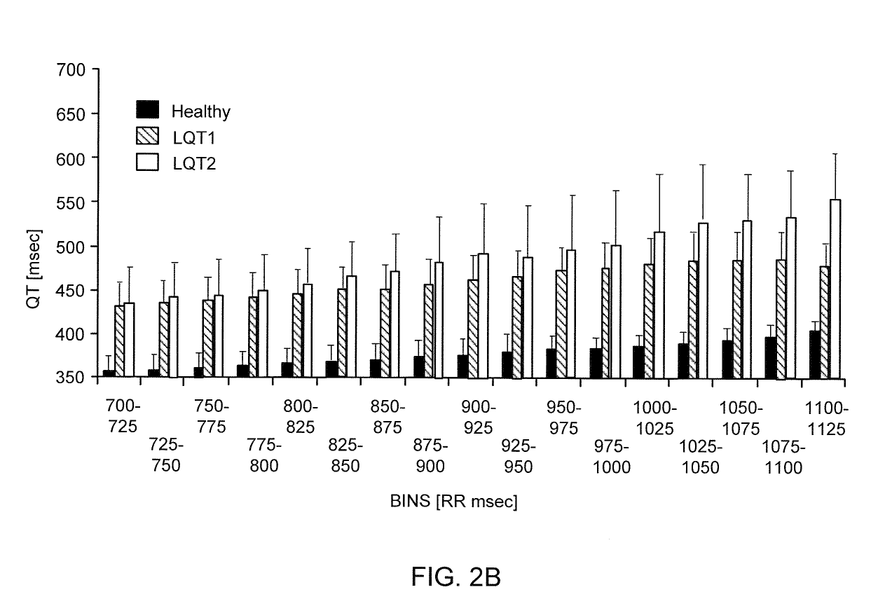

The RR bin methods is inspired from the work of Badilini et al, in their publication entitled “QT Interval Analysis on Ambulatory Electrocardiogram Recordings: A Selective Beat Averaging Approach” which was published in the Med. Biol. Eng. Comput 1999 January ; 37(1):71-9, and which is herein incorporated by reference. We developed a slightly different algorithm in which averaged values of QT and T-wave magnitude are computed from a set of representative beats rather than a unique one. This ensures appropriate measure in case of strong transient changes in drug-induced T-wave morphology such as the ones observed in individuals on sotalol. The representative beats (10-beat based) are selected according to their heart rate (limited RR interval range: RR bin). We investigated the repolarization values for heart rate between 86 bpm and 53 bpm using 17 bins (RR=7001125 ms by steps of 25 ms). For each bin, we computed the average values of QT duration and T-wave amplitude and the average standard deviation of these measures in each RR bin (see FIGS. 2A, 2B, 3A, and 3B).

### Statistical Analysis

Based on t-test or non-parametric Kruskal-Wallis tests, we compared means and median values between groups. A p-value <0.05 was considered significant. All analysis were realized using the SAS software (SAS Institute Inc., Cary, N.C., USA). The linear regression analysis for QT/RR and Tamp/RR models were computed. We reported the characteristics of the range of heart rate values investigated using the first quartile (Q1) and the intra quartile range (IQR) of the RR values used in the computation of the QT/RR and Tamp/RR slopes. We visually reviewed each scatterplots describing the QT/RR and Tamp1/RR in order to evidence outlying values that might have biased the modeling of the linear relationship. A linear mixed effect model was used to investigate the role the various ECG parameters in predicting the presence of sotalol. Binary logistic regression models were used to predict the type of mutations when analyzing the data from the patients with the congenital long QT syndrome.

### Results

### Population Characteristics:

The study groups consisted of 25 LQT1 carriers (age: 35.5±9.4 yrs), 49 LQT2 carriers (age: 34.3±10.2 yrs), and 37 healthy individuals (age: 27.5±8.1 yrs). The clinical characteristics of these groups are provided in Table 1.

TABLE 1
Characteristics of study population

| | Healthy | LQT1 | LQT2 |
|---|---|---|---|
| | n = 37 | n = 49 | n = 25 |
| Females | 29% | 71%† | 76%† |
| Age (yrs) | 27.5 ± 8.1 | 34.3 ± 10.2† | 35.5 ± 9.4† |
| Beta-Blockers (%) | 0 | 63 | 44 |
| RR (ms) | 784 ± 72 | 841 ± 114† | 837 ± 153 |
| T amplitude (mV) | 041 ± 0.15 | 0.42 ± 0.16 | 0.16 ± 0.17†* |
| QT (ms) | 367 ± 19 | 451 ± 37† | 470 ± 69† |
| QTc F (ms) | 399 ± 16 | 479 ± 28† | 499 ± 48† |
| QTc B (ms) | 417 ± 18 | 495 ± 28† | 515 ± 40† |

Average values and standard deviations for overall diurnal period. Measurements are from lead II. QTc B: heart-rate corrected QT using Bazett's formula; QTc F: QTc corrected using Fridericia formula.
†p < 0.02 in comparison to healthy group.
*p < 0.05 in reference to LQT1.

There were more females in LQT1 and LQT2 groups than in a cohort of healthy subjects. Thirty-one LQT1 and 11 LQT2 carriers were on beta blockers at the time of the recording of the Holter ECG. Mean QTc (shown for diurnal period) was significantly longer in LQTS subjects in comparison to healthy controls. There was a trend toward a longer QTc in LQT2 than LQT1 subjects. Mean T wave amplitude was significantly lower in LQT2 carriers in comparison to LQT1 carriers and healthy subjects

The number of ECGs recorded in the group healthy subjects was reduced in the double sotalol dose aim. All 10 females were excluded because of the large QT prolongation measured at a single dose. Consequently, 21 subjects were included in the second part of the experiment. As shown in Table 2, QTc was progressively prolonged after increasing dose of sotalol. Mean levels of T wave amplitude were not significantly different when comparing values off and on sotalol.

TABLE 2
Average effect of sotalol on repolarization
duration and amplitude in healthy subjects.

| | Off drug | Low dose | High dose |
|---|---|---|---|
| | n = 37 | n = 37 | n = 21 |
| RR (ms) | 784 ± 72 | 893 ± 63† | 947 ± 70†* |
| T-amplitude (mV) | 041 ± 0.15 | 0.41 ± 0.15 | 0.36 ± 0.12 |
| QT (ms) | 367 ± 19 | 405 ± 23† | 427 ± 24†* |
| QTc F (ms) | 399 ± 16 | 422 ± 22† | 437 ± 20†* |
| QTc B (ms) | 417 ± 18 | 431 ± 24† | 441 ± 20† |

Average values and standard deviations for overall diurnal period between individuals. Measurements are from lead II. QTc B: heart-rate corrected QT using Bazett's formula; QTc F: QTc corrected using Fridericia formula,
†p < 0.01 in comparison to healthy group.
*p < 0.05 in reference to low dose group

### RR Bin Analysis: QT Interval and T-Wave Amplitude Across HR:

The RR bin analysis of the QT interval duration is summarized in FIGS. 2A and 2B. It demonstrates the presence of significant QT interval prolongation in patients with the LQT1 and LQT2 as well as in individuals on sotalol in comparison to healthy individuals. In the congenital LQTS (FIG. 1B), we found a significant difference of QT interval duration (540±53 vs. 492±31 ms, p<0.05) between LQT1 and LQT2 patients only at low heart rate for RR>1075 msec. In the sotalol-induced form of the syndrome, the dose-dependent effect of sotalol on QT interval duration is also occurring mainly at low-heart rate but this trend did not reach statistical significance.

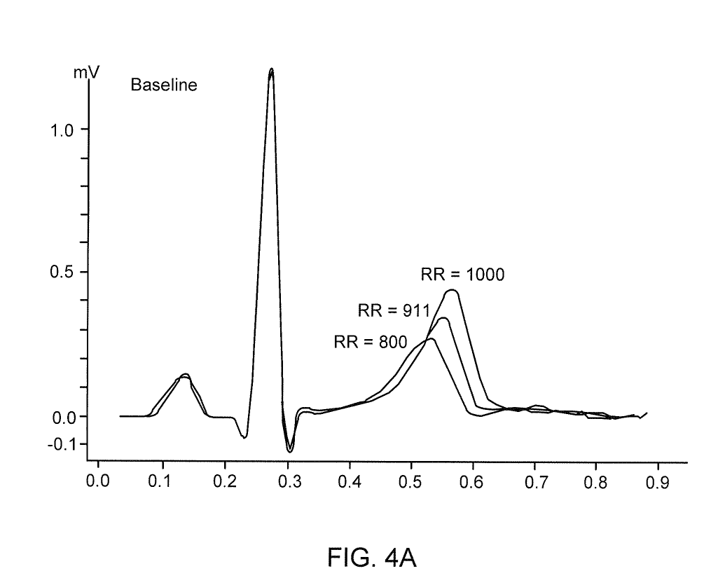

In FIGS. 3A and 3B, the values of T-wave amplitude are shown across the RR bins. In the congenital LQTS patients, no difference was found between LQT1 and healthy individuals whereas LQT2 patients showed strong significant decrease of T-wave amplitude leading to a loss of the relationship between T-amplitude and RR bin. The effect of sotalol on T-wave amplitude shows very similar pattern with a clear dose-dependent effect of the drug on T-wave amplitude exacerbated at low heart rate. The example of the effect of heart rate and sotalol on the surface ECG is illustrated in FIGS. 4A-4C.

### Repolarization and HR Dependency:

QT/RR relationship: The analysis of QT/RR in healthy subjects (Table 3) showed a linear slope equal to 0.12±0.04 in healthy subjects and a significantly higher slope in LQT1 and LQT2 carriers (QT slope >0.17).

TABLE 3
Repolarization parameters and heart rate dependency in studied groups

| | | | | Low dose | High dose |
|---|---|---|---|---|---|
| | Healthy | LQT1 | LQT2 | Sotalol | Sotalol |
| | N = 37 | N = 49 | N = 25 | N = 37 | N = 21 |
| Q1 RR (ms) | 700 ± 64 | 764 ± 94 | 771 ± 129 | 810 ± 64 | 864 ± 66 |
| IQR RR (ms) | 165 ± 42 | 151 ± 76 | 123 ± 53 | 162 ± 48 | 162 ± 30 |
| Nb beats | 5393 ± 459 | 3000 ± 910 | 2584 ± 955 | 4790 ± 410 | 4526 ± 320 |
| QT/RR slope | 0.12 ± 0.04 | 0.17 ± 0.10* | 0.22 ± 0.16* | 0.15 ± 0.05* | 0.14 ± 0.06 |
| Tamp/RR | 0.55 ± 0.29 | 0.62 ± 0.40 | 0.31 ± 0.27*‡ | 0.26 ± 0.19*‡ | 0.21 ± 0.14*‡ |
| (μV/ms) slope | | | | | |

p < 0.05 in reference to healthy,
‡p < 0.05 in reference to LQT1,
Q1: first quartile and IQR: intra quartile range
Nb beats: average number of representative beats for the calculation of the regression slopes.

The effect of sotalol on the QT/RR relationship was much less pronounced with values equal to 0.15±0.05 and 0.14±0.06 for single and double dose, respectively. It is noteworthy that the QT/RR slope was not associated with sotalol dose-dependent changes.

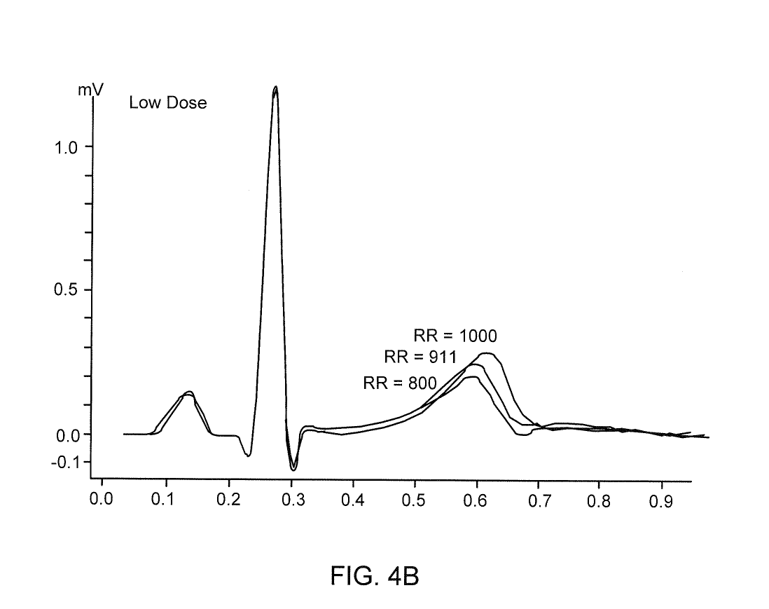

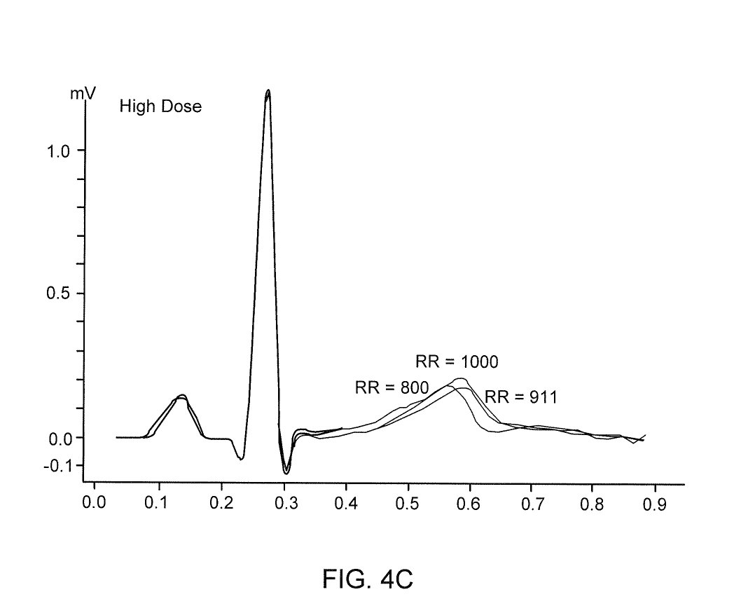

FIG. 5A provides a schematic presentation of the QT/RR relationship for all groups.

T-amplitude/RR relationship: The value of the slope characterizing the relationship between the amplitude of the Twave and the RR intervals was 0.55±0.29 pV/ms in healthy individuals. In Holter recordings of patients with the congenital form of the LQTS, no changes in T-amplitude/RR slope was observed in LQT1. However, a significantly decreased slope was found in LQT2 (0.31±0.27 pV/ms) in comparison to healthy individuals (p<0.05). We also found a dose-dependent effect on the slope of the T-wave amplitude/RR relationship in the ECGs of individuals on sotalol. The slope on single dose was 0.26±0.19 pV/ms and 0.21±0.14 pV/ms (p<0.05). The T amplitude/RR relationship for all investigated groups is provided in FIG. 5B.

This retrospective study investigates parameters of repolarization dynamics in conditions reflecting congenital and acquired abnormality of IKr function in comparison to control recordings in healthy subjects. The analysis confirmed the increased steepness of the QT/RR slope in individuals with the LQT1 and LQT2 as well as in healthy subjects on sotalol. In LQT2 patients, the QT interval was longer at slower heart rate leading to a steeper QT/RR slope than in LQT1 and healthy subjects. These results confirm previous results on the heart rate dependency of QT interval in subtype of the congenital LQTS and the prominent role of IKr current at slow heart rate. The T-amplitude/RR relationship was found to be significantly reduced in patients with the LQT2 syndrome and on sotalol in comparison to healthy individuals and LQT1 patients. This preliminary observation related to abnormal dynamics of the amplitude of the T-wave in individual with IKr related repolarization has not previously been reported. Following our observations, this factor may be useful for improving the diagnosis of congenital LQTS because of its phenotype/genotype correlation and also for the assessment of drug cardiotoxicity specific to Ikr blockade.

### T-Amplitude and Heart Rate Dependency

The genesis of the T-wave on the surface ECG remains to be fully elucidated but the current understanding recognizes the role of the sum of all existing local differences in repolarization forces within the ventricles to be the primary source for this signal (dipolar theory). There are clinical findings demonstrating that the T wave may also have non-dipolar content as well. There is a combined contribution of local and global repolarization forces to the T wave on the body surface that may significantly vary between clinical situations. Based on a transmural wedge preparation, it has also been demonstrated that transmural dispersion of myocardium also may play a role in the morphology and duration of the T wave. The disparity of the repolarization properties of the cells across the myocardium (epicardium, middle and endocardium cells) is recognized as the primary mechanism for the transmural voltage gradient recorded during the ventricular repolarization process of the heart. It is also believed that a reduction of transmural repolarization gradient is associated with a lower T-wave amplitude, it has been shown in patient with dilated cardiomyopathy: the amplitude of T-wave is significantly lowered than in healthy individuals. Despite this progress in the understanding of the role of various cells inside the myocardium, the genesis of the T-wave on the surface ECG remains a debated discussion and a very limited number of studies have investigated the changes of T-wave amplitude across heart rate when measured from surface ECGs.

It is noteworthy that most studies looking at the relationship between T-amplitude and RR interval have focus on post-exercise period and evidence a relationship between the amplitude of the T-wave and RR interval that was inversely proportional. In our investigation, the T-wave signals are analyzed during repolarization steady state avoiding the period when repolarization adapts to heart rate changes. The measurement of the amplitude of T-wave is controlled for heart rate through the RR bin technique. Interestingly, the relationship of T-amplitude and RR interval is inverted in comparison to non-steady state with increased amplitude during slower heart rate (RR increased). This observation is most likely explained by two very different electrophysiological mechanisms. At repolarization steadystate, a low heart rate is associated with an increased vagosympathetic balance of the autonomic regulation of the heart. The parasympathetically-driven balance is known to alter the acetylcholine-sensitive potassium channel (decreased catecholamine level) and to inhibit adrenergic action leading to respectively, an increased transmembrane flux of potassium ions (Ito, IK) and a reduction of outward ion currents involved in the formation of the phase 0 and 1 of the action potential (Ica++, and INa+. Because potassium ion currents (ITo, IK) are known to have longer potential duration in endocardial myocytes than epicardial human heart, one may expect a positive relationship between QT interval and RR as well as between T-amplitude and RR interval. On the other hand, just after exercise (i.e. during repolarization non-steady state), several additional factors may play a role in the inversion to this T-amplitude relationship: a depletion of repolarization reserve, presence of hyperventilation leading to alkalosis (and hypokalemia) and increased blood pressure. All these factors may lead to decrease potassium plasma concentration and thus reduced the voltage ventricular gradient (and reduced T-wave amplitude) even if the vagally-driven regulation of the heart tends to increase transmembrane potassium flux.

The extrapolated effects of changes in ion kinetics inside the myocardium to the morphology of the surface ECGs is fully hypothetical and can be strongly challenged by the inverse-problem of electrocardiography. Nevertheless, a positive relationship between heart rate and T-wave amplitude at steady state is consistent with current understanding of the cycle-dependent of IKs kinetics. The M cells are known to have much more prominent cycle length dependency of their action potentials than the epi- and endocardial myocardial cells as described by simulated study in Viswanathan et al in a publication entitled, “Effects of IKr and Iks heterogeneity on action potential duration and its rate dependence: a simulation study, published in Circulation, 1999 May 11; 99(18):2466-74. Thus, leading to a reduced voltage gradient across the myocardium at higher heart rate reflected by a positive relationship between RR and both QT interval and T-wave amplitude on the surface ECGs.

### Ikr Blockade and T-Wave Amplitude

The clinical observation described in this work on the effect of IKr blocking compound on the amplitude of T-wave is not consistent with current work on the modeling of ionic current basis of electrocardiographic waveform, such as that described in a publication entitled, “Ionic Current Basis of Electrocardiographic Waveforms: A Model Study”, published in Circ. Res. 2002 May 3; 90(8):889-96, by Gima et al. Gima et al. reports an increased T-wave amplitude following a blockade of the IKr ion currents when modeling the surface ECG in LQT2 mutation. The explanation for this inconsistency is unclear but one may hypothesize that myocardial cells in LQT2 go through remodeling where the lack of IKr kinetics is compensated by the slow component of the potassium current, another alternative resides in the lack of understanding of the cycle-dependency of IKr kinetic in human cardiac cells.

The changes in the T-amplitude/RR slope are much more pronounced than the changes of QT/RR slope. The largest changes in QT/RR slope was found in LQT2 patients with a 1.8 fold increased in steepness, the other groups showed an averaged 1.3 fold increase only, whereas the T-amplitude/RR slope in individuals with abnormal Ikr kinetics have a systematic 2-fold decrease in slope. We conclude that the lack of changes in T-amplitude/RR slope in comparison to healthy individuals is a common feature found in individuals with IKr inhibition. This is consistent with current studies that have investigated the effect of IKr blocking compounds or IKr related channelopathies. The work from Houltz et al investigated the predictive value of ECG parameters for almokalant-induced conversion of chronic atrial tachyarrhythmias. They reported a significant decreased of T-wave amplitude in individuals on almokalant and that early T-wave amplitude decrease was predicted the conversion to sinus rhythm. Heart rate dependency of this amplitude decrease was not studied. Several other Ikr inhibitory compounds have been associated with a reduction of the T-wave such asalmokalant but no studies have investigated the effect of this ion inhibition on the T amplitude/RR relationship or consider correcting the T-amplitude measurement for heart rate. See Houltz et al, “Electrocardiographic and Clinical Predictors of Torsades de Pointes Induced by Almokalant Infusion in Patients with Chronic Atrial Fibrillation or Flutter: A Prospective Study”, published in Pacing Clin. Electrophysiol 1988 May; 21(5):1044-57 and Houltz et al, “Effects of the IKr-Blocker Almokalant and Predictors of Conversion of Chronic Atrial Tachyarrhythmias to Sinus Rhythm: A Prospective Study”, published in Cardiovasc. Drugs Ther. 1999 July; 13(4):329-38.

### Further Conclusions

This is a retrospective observational study involving individual with both the congenital and acquired form of the LQTS. The T-wave amplitude is an electrocardiographic parameters that can be affected by the body position, the lead placement. One may safely state that it is less true for the QT interval measurements. Nevertheless, our work demonstrate that an IKr blockade affect the amplitude but more importantly the dynamic mechanisms of the T-wave amplitude. We believe that such observation is truly important because in such analysis each individual become its own reference and there is a dose-dependent effect (based on sotalol observation). The application of such electrocardiographic phenomenon may play an important role in the future of assessment of both the penetrance of KCNH2-mutation and the Ikr related cardiotoxicity of drugs

This work demonstrates the presence of a relationship between the T-wave amplitude and the heart rate. An impaired adaptation of T-wave amplitude has been shown as common electrocardiographic feature associated with KCNH2 mutation and Ikr blocking drug sotalol. This ECG marker may play an important role in the future of assessment of both the penetrance of KCNH2-mutation and the Ikr related cardiotoxicity of drugs

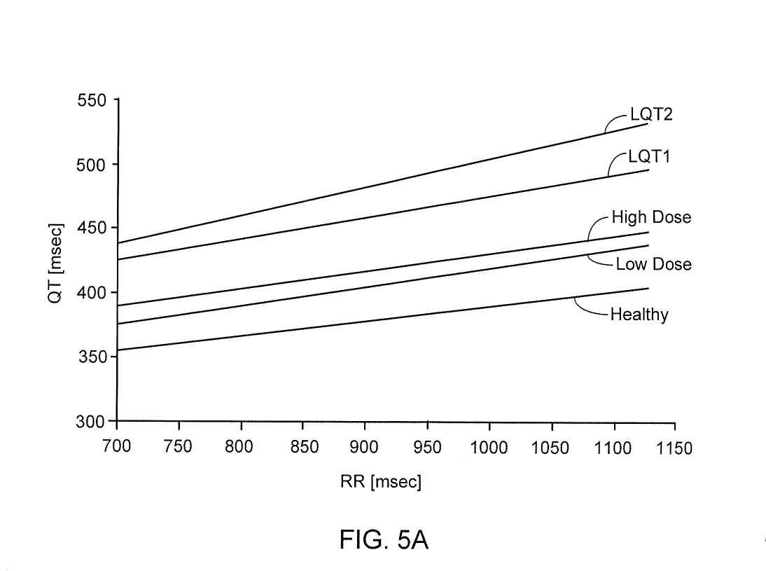

FIG. 6 illustrates one embodiment of a method for assaying impaired delayed rectifier potassium current (IKr) function. One or more electrocardiogram (ECG) signals are obtained 40. The one or more ECG signals may be obtained from a variety of ECG capture devices as discussed above. The one or more ECG signals may be obtained in “real-time” from one or more subjects, or the one or more ECG signals may be obtained from a database (which should be understood to include memory devices) storing previously obtained ECG signals.

One or more heart rates, corresponding to one or more beats of the one or more ECG signals, are obtained 42. The heart rate can be measured concurrently as the one or more ECG signals are obtained, or the heart rates can be determined after the fact using known signal processing techniques and their equivalents to pull out the heart rate information from the one or more ECG signals.

One or more T-wave amplitudes are determined 44 corresponding to one or more beats of the one or more ECG signals. Various methods of determining T-wave amplitude are known to those skilled in the art.

Prior to determining 44 the one or more T-wave amplitudes, it may be necessary in some embodiments to filter 39 the one or more ECG signals. Some sources of ECG data may already be filtered, however, in which case this step would not be necessary. In cases where the ECG data is not pre-filtered, filtering 39 of the ECG signals is recommended to remove baseline wander in the signals, although filtering is not necessary in all embodiments. One suitable method of filtering the ECG signals to remove baseline wander is digital low-pass FIR filtering. Another suitable method of filtering the ECG signals to remove baseline wander is to subtract a baseline estimation arrived-at using spline interpolation.

In other embodiments, the filtering 39 may include statistical combinations of multiple beats from the ECG signals. As a non-limiting example, a median beat may be created from a number of consecutive beats from each lead. In some embodiments, one or more leading beats may be discarded. In other embodiments, one or more trailing beats may be discarded. In further embodiments, only beats with a stable heart rate may be taken into account. An example of a suitable definition of beats with a stable heart rate is when the heart rate for a given beat varies less than ten percent in beats of the previous two minutes. In other embodiments other percentages, time-frames, and definitions of a stable heart rate may be used without deviating from the scope of the claimed invention.

Once the one or more T-wave amplitudes are determined 44, a determination can be made 46 as to whether the one or more T-wave amplitudes as a function of the one or more heart rates are relatively independent of the corresponding one or more heart rates, thereby indicating impaired delayed rectifier potassium current function. This comparison can be done graphically, or by a slope comparison as demonstrated above with regard to the experimental data. T-wave amplitude may be plotted or compared with heart rate by bin method or by direct correlation to heart rate without using bins. If using a bin method, a statistical analysis of T-wave amplitude can be used to provide, for example, an average T-wave amplitude within each heart rate bin. Indications of statistical deviation and variance can be included as desired. Additionally, a biomarker indicative of IKr impairment may be derived from the T-wave amplitudes and corresponding heart rate data. Examples of such a biomarker include, but are not limited to 1) a biomarker indicating the linearity of the T-wave amplitude versus heart rate data; and 2) a biomarker indicating the slope of the T-wave amplitude versus heart rate data.

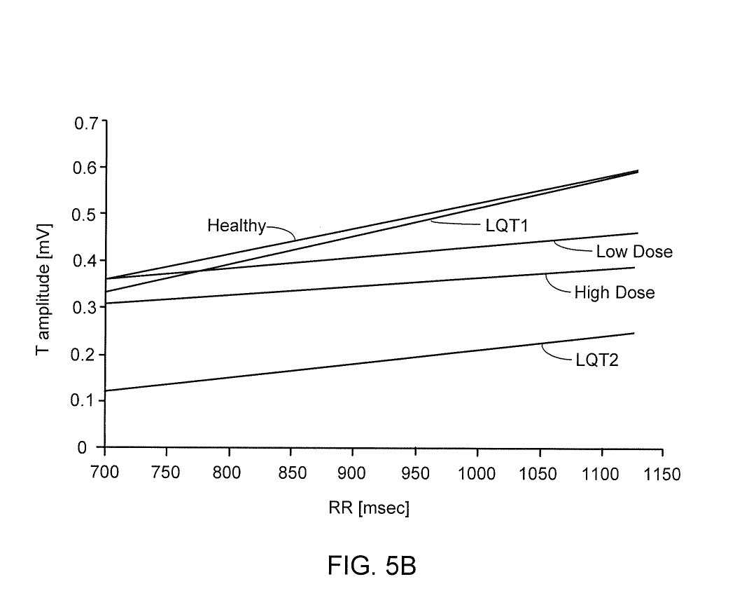

FIG. 7 schematically illustrates an embodiment of a system 60 for assaying impaired delayed rectifier potassium current (IKr) function. The system has a processor 62 which is configured to determine whether the T-wave amplitude of the electrocardiogram as a function of heart rate is relatively independent of heart rate. Embodiments of suitable processes and method steps to make this determination have already been discussed above. The processor 62 may be a computer executing machine readable instructions which are stored on a CD, a magnetic tape, an optical drive, a DVD, a hard drive, a flash drive, a memory card, a memory chip, or any other computer readable medium. The processor 62 may alternatively or additionally include a laptop, a microprocessor, an application specific integrated circuit (ASIC), digital components, electrical components, or any combination thereof. The processor 62 may be a stand-alone unit, or it may be a distributed set of devices.

A data input 64 is coupled to the processor 62 and configured to provide the processor with ECG data. An ECG capture device 66 may optionally be coupled to the data input 64 to enable the live capture of ECG data. Examples of ECG capture devices include, but are not limited to, a twelve-lead ECG device, an eight-lead ECG device, a two lead ECG device, a Holter device, a bipolar ECG device, and a uni-polar ECG device. Similarly, a database 68 may optionally be coupled to the data input 64 to provide previously captured ECG signal data to the processor. Database 68 can be as simple as a memory device holding raw data or formatted files, or database 68 can be a complex relational database. Depending on the embodiment, none, one, or multiple databases 68 and/or ECG capture devices 66 may be coupled to the data input 64. The ECG capture device 66 may be coupled to the data input 64 by a wired connection, an optical connection, or by a wireless connection. Suitable examples of wireless connections may include, but are not limited to, RF connections using an 802.11x protocol or the Bluetooth® protocol. The ECG capture device 66 may be configured to transmit data to the data input 64 only during times which do not interfere with data measurement times of the ECG capture device 66. If interference between wireless transmission and the measurements being taken is not an issue, then transmission can occur at any desired time. Furthermore, in embodiments having a database 68, the processor 62 may be coupled to the database 68 for storing results or accessing data by bypassing the data input 64.

The system 60 also has a user interface 70 which may be coupled to either the processor 62 and/or the data input 64. The user interface 70 can be configured to display the ECG signal data, the T-wave amplitude plotted as a function of heart rate as discussed above, and/or calculated parameters such as the slope of the T-wave amplitude versus heart rate. Other indications of T-wave dependency or lack thereof on heart rate may be displayed by the user interface 70, depending on the embodiment. The user interface 70 may also be configured to allow a user to select ECG signals from a database 68 coupled to the data input 64, or to start and stop collecting data from an ECG capture device 66 which is coupled to the data input 64.

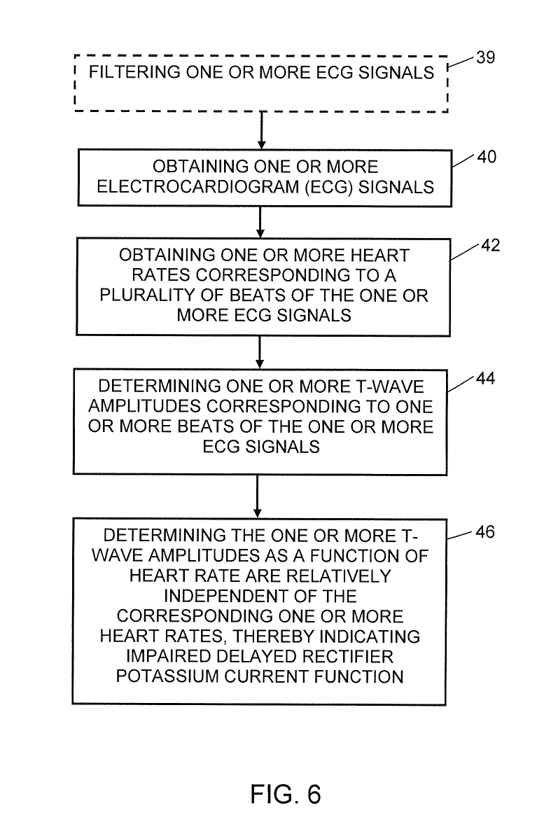

FIG. 8 schematically illustrates another embodiment of a system 72 for assaying impaired delayed rectifier potassium current (IKr) function. In this embodiment, the processor 62 is set-up to be a remote processor which is coupled to the data input 64 over a network 74. The network 74 may be a wired or wireless local area network (LAN or WLAN) or the network 74 may be a wired or wireless wide area network (WAN, WWAN) using any number of communications protocols to pass data back and forth. Having a system 72 where the processor 62 is located remotely allows multiple client side data inputs 64 to share the resources of the processor 62. ECG signals may be obtained by the data input 64 from a database 68 and/or an ECG capture device 66 under the control of a user interface 70 coupled to the data input 64. The ECG signal data may then be transferred over the network 74 to the processor 62 which can then determine whether the T-wave amplitude of the ECG data as a function of heart rate is relatively independent of heart rate and transmit data signals 76 having such conclusion and/or related biomarker parameters to the client side. Such data transmissions may take place over a variety of transmission media, such as wired cable, optical cable, and air. In this embodiment, the remote processor 62 can be used to help keep the cost of the client-side hardware down, and can facilitate any upgrades to the processor or the instructions being carried out by the processor, since there is a central upgrade point.

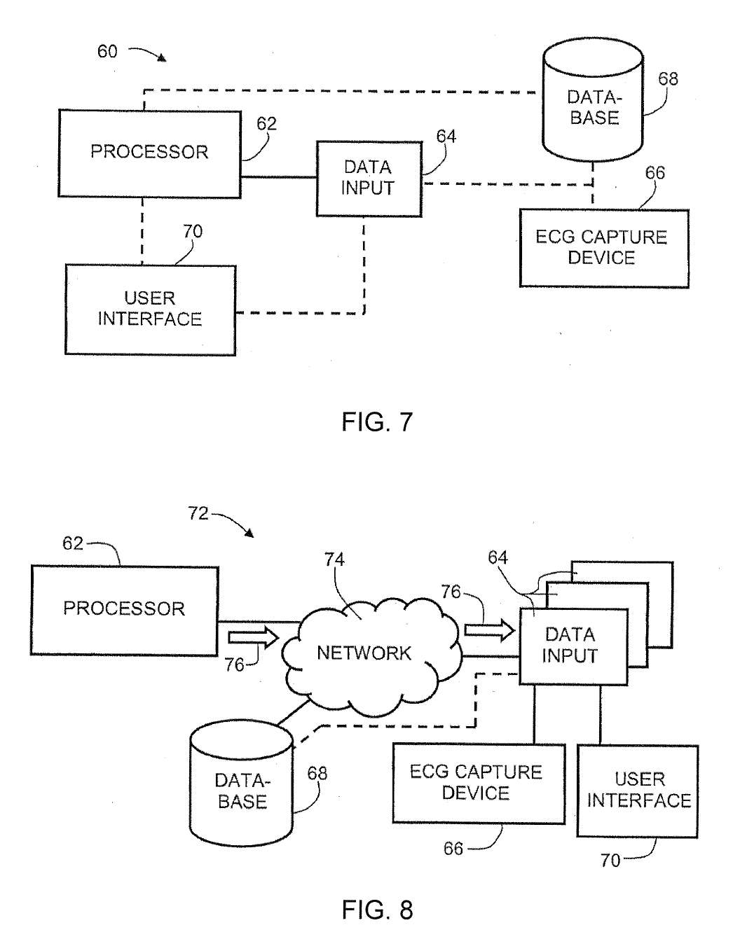

FIG. 9 schematically illustrates a further embodiment of a system 78 for assaying impaired delayed rectifier potassium current (IKr) function. In this embodiment, a data input 64, a user interface 70, and a database 68 are coupled to the processor 62. An ECG capture device 66 is coupled to the data input 64. The system 78 also has a pharmacological agent administrator 80 which is coupled to the processor 62. The pharmacological agent administrator 80 may be configured to administer a pharmacological agent to a patient when enabled by the processor 62. The system 78 of FIG. 9, and its equivalents, may be useful in automating the analysis of the effects of pharmacological agents on heart repolarization. A baseline determination of whether the T-wave amplitude of the ECG as a function of heart rate is relatively independent of heart rate. Then, the processor can instruct the pharmacological agent administrator 80 to administer a pharmacological agent. Then, a follow-on determination of whether the T-wave amplitude of the ECG as a function of heart rate is relatively independent of heart rate. Changes in the dependency of the T-wave amplitude on heart rate may then be determined to be indicative of an interaction between the pharmacological agent and the delayed rectifier potassium current function.

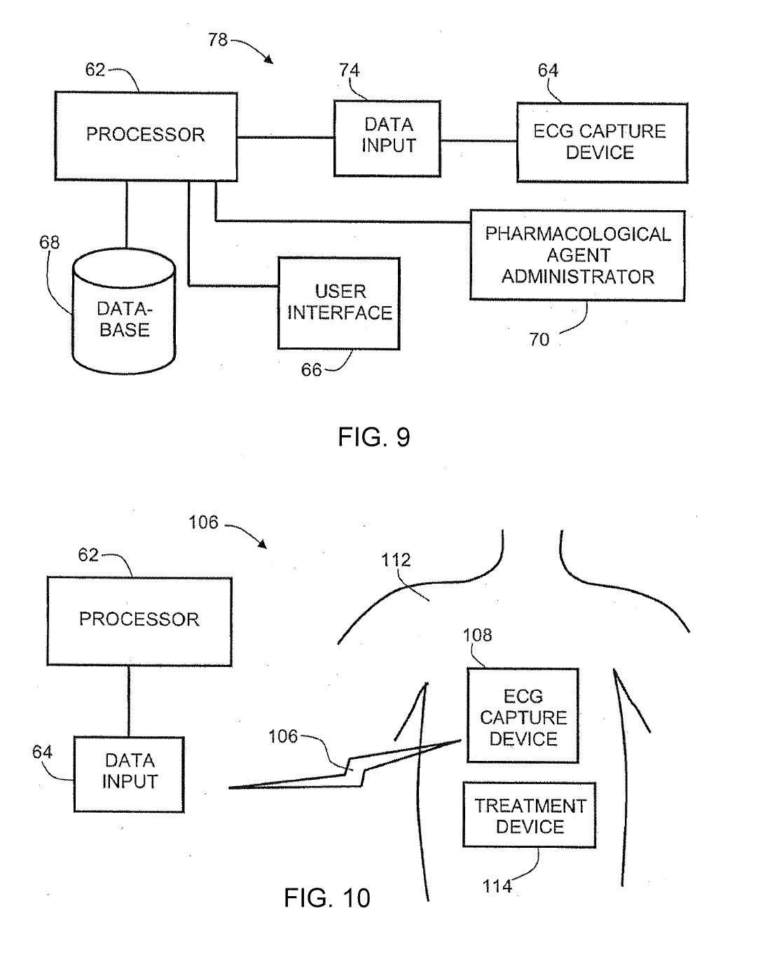

FIG. 10 schematically illustrates another embodiment of a system 106 assaying the impaired delayed rectifier potassium current function. Similar to other embodiments, the system has a processor 62 which is coupled to a data input 64. An ECG capture device 108 is coupled 110 to the data input. The coupling 110 may be wired or wireless. The ECG capture device 108 is configured so that at least a portion of the ECG capture device 108 is implantable in a subject's body 112. The processor 62 and the data input 64 are external to the subject's body 112 in this embodiment, however, in other embodiments, the processor 62 and/or the data input 64 could be partially or entirely implanted in the subject's body 112. The system 106 of FIG. 10 may optionally have a treatment device 114 coupled to the processor 62. In this case, the processor 62 may be configured to activate the treatment device to attempt to compensate for an impaired delayed rectifier potassium current function as determined by one of the methods above. Suitable examples of treatment devices 114 include, but are not limited to a pharmacological agent administrator and a defibrillator. The treatment device 114 may also be partially or completely implanted inside of the subject 112.

The advantages of a method and system to identify impaired IKr kinetics based on loss of heart rate dependency of the T-wave amplitude have been discussed herein. Embodiments discussed have been described by way of example in this specification. It will be apparent to those skilled in the art that the forgoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A method of assaying impaired delayed rectifier potassium current function, comprising:
obtaining one or more electrocardiogram (ECG) signals;
obtaining one or more heart rates corresponding to a plurality of beats of the one or more ECG signals;
determining one or more T-wave amplitudes corresponding to one or more beats of the one or more ECG signals; and
determining whether the one or more T-wave amplitudes as a function of the one or more heart rates are relatively independent of the corresponding one or more heart rates, thereby indicating impaired delayed rectifier potassium current function.

2. The method of claim 1, further comprising:
filtering the one or more ECG signals.

3. The method of claim 2, wherein filtering one or more ECG signals comprises low-pass FIR filtering the one or more ECG signals.

4. The method of claim 2, wherein filtering the one or more ECG signals comprises removing a wandering baseline.

5. The method of claim 2, wherein filtering the one or more ECG signals comprises statistically combining multiple beats from the one or more ECG signals.

6. The method of claim 2, wherein filtering the one or more ECG signals comprises discarding one or more leading beats from the one or more ECG signals.

7. The method of claim 2, wherein filtering the one or more ECG signals comprises discarding one or more trailing beats from the one or more ECG signals.

8. The method of claim 2, wherein filtering the one or more ECG signals comprises discarding beats which do not have a corresponding stable heart rate.

9. The method of claim 8, wherein discarding beats which do not have a corresponding stable heart rate comprises discarding beats which have a heart rate that varies by more than a certain percentage in a previous arbitrary time frame.

10. The method of claim 1, wherein determining whether the one or more T-wave amplitudes as a function of the one or more heart rates are relatively independent of the corresponding one or more heart rates comprises:
sorting the one or more T-wave amplitudes into heart rate bins; and
calculating an average T-wave amplitude for each of the heart rate bins.

11. The method of claim 10, further comprising determining a slope of the average T-wave amplitude versus the heart rate bins.

12. The method of claim 1, wherein determining whether the one or more T-wave amplitudes as a function of the one or more heart rates are relatively independent of the corresponding one or more heart rates comprises plotting the one or more T-wave amplitudes as a function of the corresponding one or more heart rates.

13. The method of claim 12, further comprising determining a slope of the plotted one or more T-wave amplitudes as a function of the corresponding one or more heart rates.

14. A biomarker produced using the method of claim 1.

15. A computer readable medium having stored thereon instructions for assaying impaired delayed rectifier potassium current function, which, when executed by a processor, causes the processor to perform the steps according to claim 1.

16. A system for assaying impaired delayed rectifier potassium current function, comprising:
a processor configured to determine an indication of impaired delayed rectifier potassium current function based on a comparison of T-wave amplitudes from ECG data with corresponding heart rates for the T-wave amplitudes;
a data input coupled to the processor and configured to provide the processor with the ECG data; and
a user interface coupled to either the processor or the data input.

17. The system of claim 16, wherein the processor is configured to be at least partially implantable in a subject's body.

18. The system of claim 16, further comprising a database coupled to the processor.

19. The system of claim 16, further comprising a database coupled to the data input.

20. The system of claim 16, further comprising an ECG capture device coupled to the data input.

21. The system of claim 20, wherein the ECG capture device is selected from the group consisting of a Holter monitor; a twelve-lead monitor; an 8 lead monitor; a monitor using a bipolar lead system, and a monitor using a unipolar lead system.

22. The system of claim 20, further comprising a treatment device coupled to the processor, and wherein the processor is further configured to activate the treatment device to attempt to correct a repolarization abnormality indicated by the impaired delayed rectifier potassium current function.

23. A method for determining the ability of a pharmacological agent to impair delayed rectifier potassium current function, comprising:
obtaining a baseline electrocardiogram (ECG) for a mammal at different heart rates;
determining a baseline dependency of a plurality of T-wave amplitudes on corresponding heart rates for the baseline ECG;
administering the pharmacological agent to the mammal;
obtaining a follow-on ECG for the mammal at different heart rates;
determining a follow-on dependency of a plurality of T-wave amplitudes on corresponding heart rates for the follow-on ECG; and
comparing the follow-on dependency with the baseline dependency.

24. The method of claim 23, wherein:
the baseline dependency is expressed as a baseline slope;
the follow-on dependency is expressed as a follow-on slope; and
comparing the follow-on dependency with the baseline dependency comprises determining whether the follow-on slope is significantly greater than the baseline slope, thereby indicating that the pharmacological agent has impaired the delayed rectifier potassium current function.
